(19) United States
(12) Patent Application Publication
(10) Pub. No.: US 2019/0014972 A1
(43) Pub. Date: Jan. 17, 2019

(54) OPERATION UNIT

(71) Applicant: OLYMPUS CORPORATION, Tokyo (JP)

(72) Inventors: Keisuke HATANO, Tokyo (JP); Kiwamu FUJITANI, Orefield, PA (US)

(73) Assignee: OLYMPUS CORPORATION, Tokyo (JP)

(21) Appl. No.: 15/651,520

(22) Filed: Jul. 17, 2017

(51) Int. Cl. A61B 1/005; A61B 1/00

(52) U.S. Cl. CPC A61B 1/0052; A61B 1/00158; A61B 1/0057

(57) ABSTRACT

An operation unit of an endoscope includes an insertion section that includes a bending portion, a plurality of operation wires configured to bend the bending portion, an operation lever that is provided on a freely tiltable operation section provided continuously to the insertion section, a wire pulling member swingably provided inside the operation section and having a plurality of arm portions, one end of the operation lever being coupled to the wire pulling member, a housing that is fixedly provided on the operation section, and that includes a through hole where the operation lever is arranged, a magnet that is provided on one of the housing and the other end portion of the operation lever, and a member on which attraction is generated by a magnetic force of the magnet, the member being provided on the other end portion of the operation lever or on the housing.

### BACKGROUND OF THE INVENTION

### 1. Field of the Invention

The present invention relates to an operation unit configured to pull a desired operation wire by a tilt operation of an operation lever.

### 2. Description of the Related Art

In recent years, endoscopes have been used in medical and industrial fields. Some endoscopes are provided with an elongated, flexible insertion section. A bending portion is provided on a distal end side of the flexible insertion section. Generally, the bending portion is bent by a user operating, on a hand side, a bending operation member provided on an operation section.

With an endoscope provided with the bending portion, an observation direction of an observation optical system provided on the distal end portion on the insertion section distal end side of the bending portion may be changed by bending the bending portion, and examination may be performed over a wide range.

For example, as disclosed in Japanese Patent Application Laid-Open Publication No. 62-38411 or Japanese Patent Application Laid-Open Publication No. 2009-89955, a conventional endoscope is provided with a lever-type or joystick-type operation member at the operation section. The bending portion is configured to be bent by pulling/slackening of an operation wire by hand-side operation of the operation member.

However, the bending portion is provided with a plurality of bending pieces that are continuous to one another in a rotatable manner, and an elastic bending rubber that covers the bending pieces. Therefore, according to a conventional endoscope as disclosed in Japanese Patent Application Laid-Open Publication No. 62-38411 or Japanese Patent Application Laid-Open Publication No. 2009-89955, the amount of force for operating the operation member is increased by the bending operation, that is, as the bending angle of the bending portion is increased.

Accordingly, a conventional endoscope has a problem that the bending operation makes a user tired, or that a fine bending operation is difficult.

An object of the present invention is to provide an endoscope which allows the amount of operational force for an operation member which is configured to bend a bending portion to be reduced so as to reduce fatigue of a user, and which allows a fine bending operation.

### SUMMARY OF THE INVENTION

An operation unit of an endoscope according to an aspect of the present invention includes an insertion section that is insertable into a subject, and that includes a bending portion that can be bent in a predetermined direction from a longitudinal axis, an operation section that is provided continuously to a proximal end of the insertion section, a plurality of operation wires that are inserted inside the insertion section and the operation section, and that are configured to bend the bending portion by being pulled or slackened, an operation lever that is provided on the operation section, relative to one end of the operation lever, other end of the operation lever being freely tiltable in the predetermined direction of the bending portion, a wire pulling member that is swingably provided inside the operation section, the one end of the operation lever being coupled to a center portion of the wire pulling member, proximal end sides of the wires being coupled to respective distal end sides of a plurality of arm portions extending sideways from the center portion, a cylindrical base section that is fixedly provided at a predetermined position of the operation section, and that includes a through hole where a mid-portion of the operation lever is arranged and an inner circumferential portion to which the other end of the operation lever comes close when the operation lever is tilted in the predetermined direction, a magnet that is provided on one of the base section and the other end of the operation lever, and a member on which attraction is generated by a magnetic force of the magnet, the member being provided on the other end of the operation lever when the magnet is provided on the base section and provided on the base section when the magnet is provided on the other end of the operation lever.

### BRIEF DESCRIPTION OF THE DRAWINGS

FIG. 1 is a front view showing an external appearance of an endoscope;

FIG. 2 is a right side view showing the external appearance of the endoscope;

FIG. 3 is a top view showing the external appearance of the endoscope;

FIG. 4 is an explanatory view showing a positional relationship between a wire pulling member and a cylinder;

FIG. 5 is a perspective view showing a positional relationship between a bending operation mechanism and the cylinder;

FIG. 6 is a perspective view showing internal structural components of the bending operation mechanism;

FIG. 7 is an exploded perspective view showing the internal structural components of the bending operation mechanism;

FIG. 8 is a transverse cross-sectional view showing main parts of a distal end portion and a bending portion;

FIG. 9 is a cross-sectional view showing the distal end portion along line IX-IX in FIG. 8;

FIG. 10 is a cross-sectional view showing the bending portion along line X-X in FIG. 8;

FIG. 11 is a cross-sectional view showing the bending portion along line XI-XI in FIG. 8;

FIG. 12 is a diagram describing an example configuration of a bending operation support mechanism configured to reduce an amount of operational force, and also describing a tilt operation of an operation lever;

FIG. 13 is a diagram describing a relationship between a tilt position of the operation lever and an amount of tilt operation force, a relationship between the tilt position and a magnetic force of a magnet, and a relationship between the tilt position of the operation lever and the amount of tilt operation force offset by attraction generated by the magnetic force;

FIG. 14A is a diagram describing another example configuration of the bending operation support mechanism configured to reduce the amount of operational force;

FIG. 14B is a diagram describing further another example configuration of the bending operation support mechanism configured to reduce the amount of operational force; and

FIG. 14C is a diagram describing still another example configuration of the bending operation support mechanism configured to reduce the amount of operational force.

### DETAILED DESCRIPTION OF THE PREFERRED EMBODIMENT

Hereinafter, an embodiment of the present invention will be described with reference to the drawings.

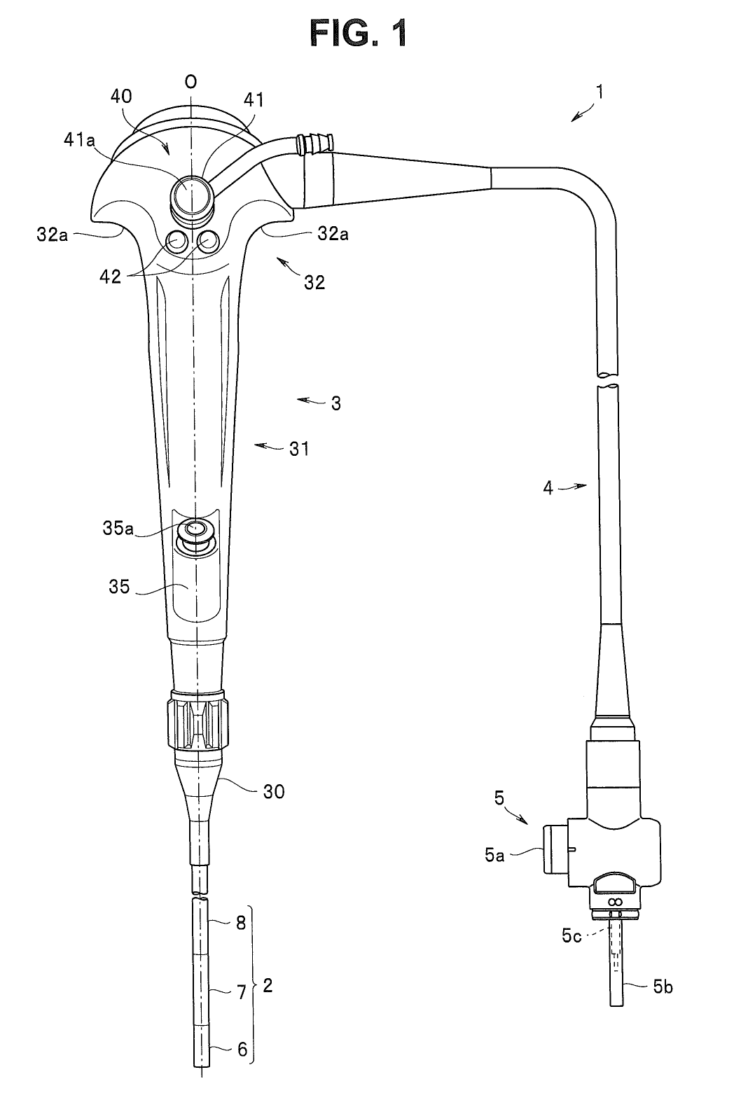

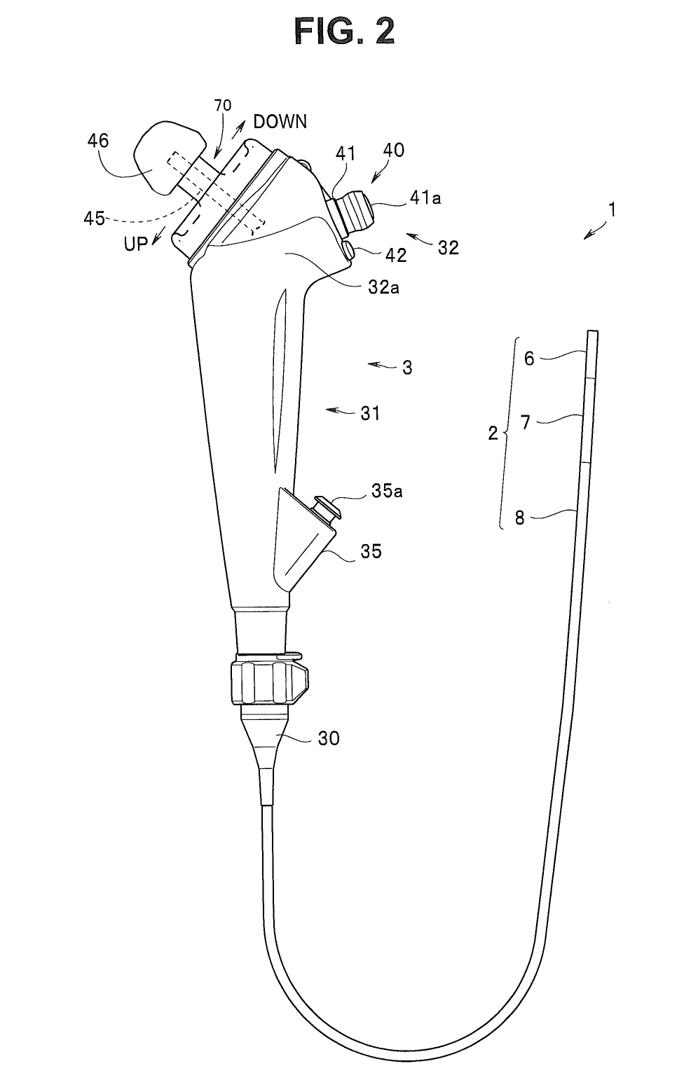

An endoscope 1 of the present embodiment shown in FIGS. 1 and 2 is an electronic endoscope for bronchus. The endoscope 1 is mainly configured from an insertion section 2, an operation section 3, and a universal cord 4. The insertion section 2 is formed as an elongated tube. The operation section 3 is continuously provided on a proximal end of the insertion section 2. The universal cord 4 is an endoscope cable, and is extended from the operation section 3. An endoscope connector 5 is disposed at an end portion of the universal cord 4.

The insertion section 2 is configured from a flexible tubular member. A distal end portion 6, a bending portion 7, and a flexible tube portion 8 are provided in a continuous manner from a distal end side of the insertion section 2.

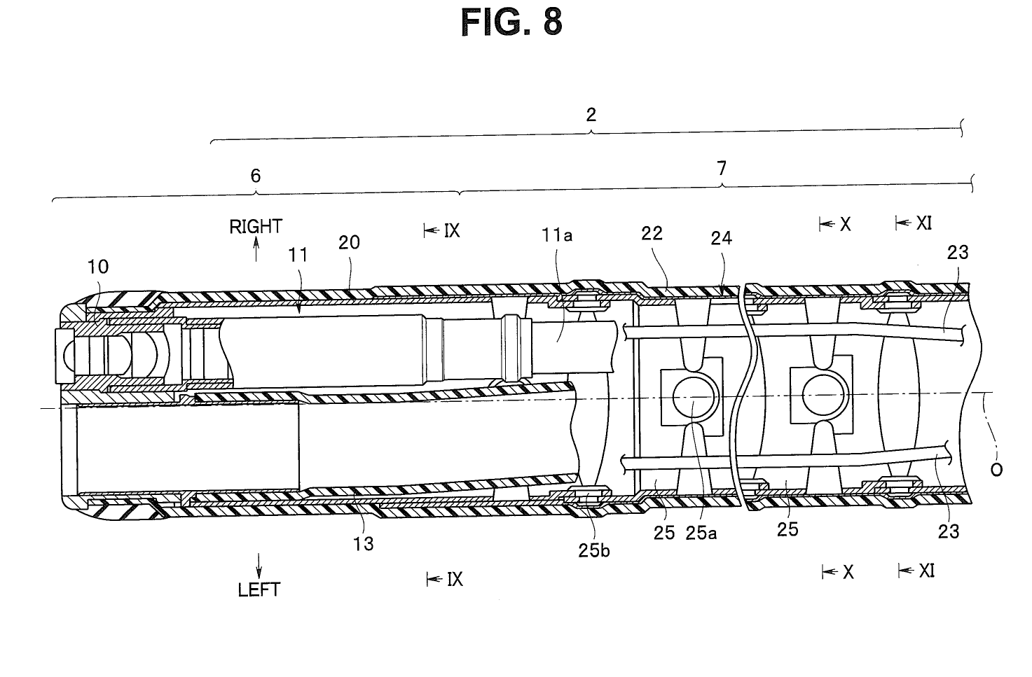

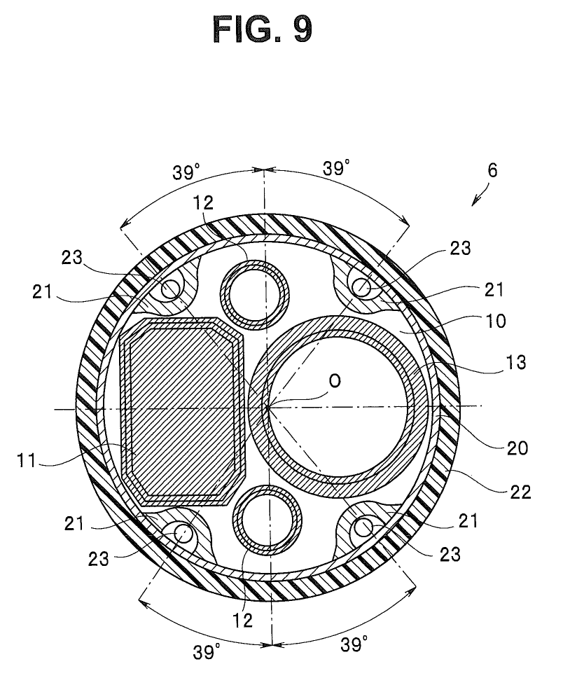

For example, as shown in FIGS. 8 and 9, a metal distal end rigid portion 10 is provided inside the distal end portion 6. An image pickup section 11 where an image pickup device such as a CCD or CMOS is built in, a pair of light guides 12, and a treatment instrument insertion channel 13 are held in the distal end rigid portion 10.

Inside the distal end portion 6, a most distal end bending piece 20 having a substantially cylindrical form is fitted at the outside of a proximal end side of the distal end rigid portion 10. An outer circumference of the most distal end bending piece 20 is covered with a bending rubber 22. Wire fixing portions 21 are provided on an inner circumference of the most distal end bending piece 20, at four positions around an insertion axis O. Any distal ends of four operation wires 23, which are inserted in the insertion section 2, are fixed to the respective wire fixing portions 21.

Furthermore, as shown in FIGS. 8 and 9, to efficiently arrange each structural member without increasing a diameter of the distal end portion 6, the image pickup section 11 and the treatment instrument insertion channel 13, which are large members, are arranged horizontally next to each other inside the distal end rigid portion 10 and the most distal end bending piece 20. The light guides 12 are arranged, respectively, in spaces at the top and the bottom formed by the arrangement described above.

Note that in the present embodiment, up-down/left-right directions of the distal end portion 6 of the insertion section 2 are directions that are defined in association with up-down/left-right directions of an image that is picked up by the image pickup section 11, for example.

Furthermore, to prevent interference of the image pickup section 11 and the treatment instrument insertion channel 13 with each operation wire 23, each of the wire fixing portions 21 is provided at a position rotated by a predetermined angle around the insertion axis O from an up, down, left or right position of the distal end portion 6. That is, as shown in FIG. 9, the wire fixing portions 21 are provided on the most distal end bending piece 20, respectively, at positions rotated to the left and the right from the up direction of the distal end portion 6, around the insertion axis O and in the range of 30 degrees to 60 degrees (more specifically, at positions rotated by, for example, 39 degrees), and at positions rotated to the left and the right from the down direction of the distal end portion 6, around the insertion axis O and in the range of 30 degrees to 60 degrees (more specifically, at positions rotated by, for example, 39 degrees), for example.

In other words, the operation wires 23 are routed inside the distal end portion 6, at positions rotated by a predetermined angle around the insertion axis O relative to the up-down/left-right directions.

The bending portion 7 is configured to be able to actively bend in any direction from the insertion axis O, including the predetermined up-down/right-left directions, according to an operation input from a user, such as a surgeon, to the operation section 3. For example, the bending portion 7 of the present embodiment includes a bending piece set 24 where a plurality of bending pieces 25 are continuously provided, the bending piece set 24 including, on a distal end sides, pivot portions 25a (see FIG. 9), which are arranged in the up-down directions of the insertion section 2, and including, on a proximal end sides, pivot portions 25b, which are arranged in the left-right directions of the insertion section 2.

Inside the bending piece set 24, a signal cable 11a extending from the image pickup section 11, the light guides 12, and the treatment instrument insertion channel 13 are inserted according to an arrangement substantially the same as the arrangement inside the distal end portion 6. An outer circumference of the bending piece set 24 is covered with the bending rubber 22 extending from a distal end portion 6 side.

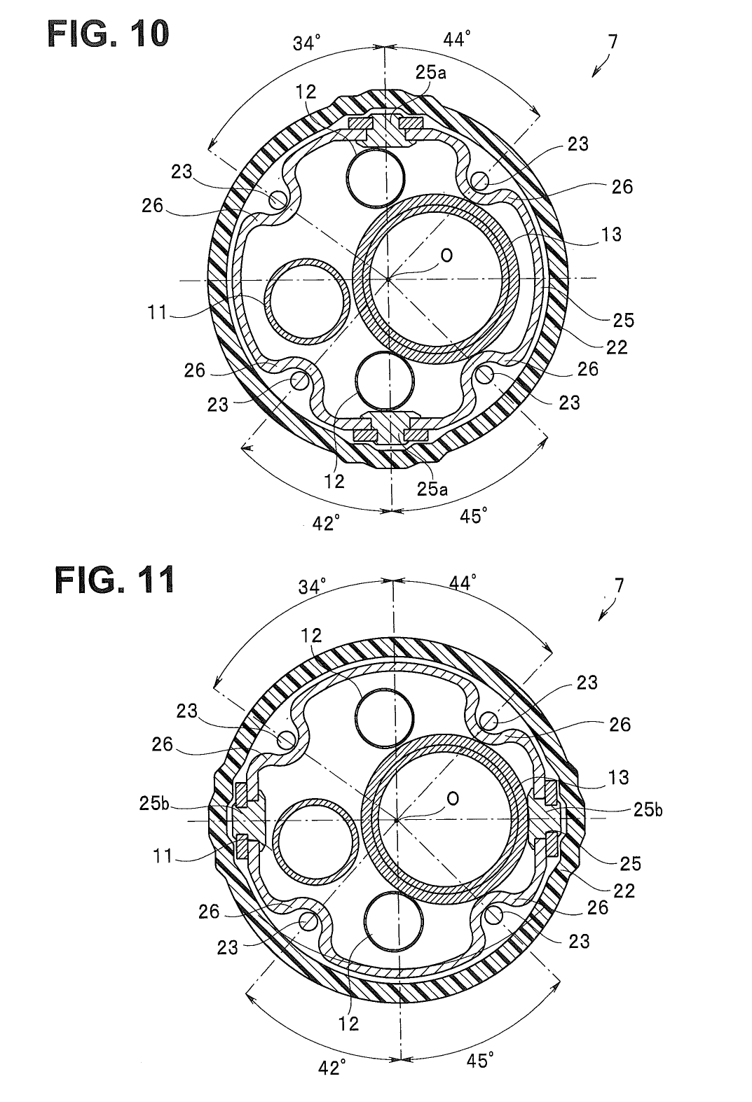

Furthermore, wire guides 26 allowing insertion of the respective operation wires 23 are formed in predetermined bending pieces 25 configuring the bending piece set 24. Like the wire fixing portions 21 described above, the wire guides 26 are provided at positions rotated by a predetermined angle around the insertion axis O from an up, down, left and right position of the bending portion 7. That is, for example, as shown in FIGS. 10 and 11, the wire guides 26 are provided in a predetermined bending piece 25, respectively at positions rotated to the left and the right in the range of 30 degrees to 60 degrees around the insertion axis O with respect to the up direction of the bending portion 7 (more specifically, for example, at positions rotated by 34 degrees to the left and 44 degrees to the right), and positions rotated to the left and the right in the range of 30 degrees to 60 degrees around the insertion axis O with respect to the down direction of the bending portion 7 (more specifically, for example, at positions rotated by 42 degrees to the left and 45 degrees to the right).

In other words, the operation wires 23 are routed inside the bending portion 7, at positions rotated around the insertion axis O relative to the up-down/left-right directions.

The flexible tube portion 8 is configured by a flexible tubular member which is capable of passively bending. The signal cable 11a, the light guides 12, and the treatment instrument insertion channel 13 described above (which are not shown in the drawing) are inserted in the flexible tube portion 8.

The operation section 3 is configured by including a bend preventing portion 30, a grasping portion 31, and an operation section main body 32. The bend preventing portion 30 is connected to the flexible tube portion 8 while covering a proximal end of the flexible tube portion 8. The grasping portion 31 is provided continuously to the bend preventing portion 30. The grasping portion 31 can be grasped by the hand of a user. The operation section main body 32 is continuously provided on a proximal end side of the grasping portion 31.

Note that in the present embodiment, directions and the like from the insertion axis O of the operation section 3 are defined relative to the state where the grasping portion 31 is grasped by the user. More specifically, front-back/left-right directions (a front surface, a back surface, left and right side surfaces, etc.) relative to the user grasping the grasping portion 31 are defined for the operation section 3.

As shown in FIG. 1, the grasping portion 31 is formed into a shape that is left-right symmetrical with respect to the insertion axis O (center axis), and can thus be grasped in the same manner by either of the left and right hands of the user.

A treatment instrument insertion portion 35 is provided on a front surface on the distal end side of the grasping portion 31. The treatment instrument insertion portion 35 includes a treatment instrument insertion opening 35a through which various treatment instruments (not shown) are inserted. Inside the operation section 3, the treatment instrument insertion opening 35a communicates with the treatment instrument insertion channel 13 via a branching member, not shown. A forceps plug (not shown), which is a lid member configured to block the treatment instrument insertion opening 35a, is freely attachable/detachable to/from the treatment instrument insertion portion 35.

The operation section main body 32 is configured, at the proximal end side of the grasping portion 31, by a hollow member having a substantially partially round shape which is expanded mainly on the left and right sides and the front side. An operation button group 40 used for realizing various functions of the endoscope 1 is provided on the front side of the operation section main body 32. An operation lever 45 configured to bend the bending portion 7 is disposed on a back side of the operation section main body 32. The universal cord 4 is extended from one side portion (for example, a left side portion) of the operation section main body 32.

The operation section main body 32 is formed expanded on the left and the right in a symmetrical manner about the insertion axis O. A guide recess portion 32a, which guides an index finger or the like of a user grasping the grasping portion 31 to the operation button group 40, is provided on each of the left and right side surfaces on the distal end side of the operation section main body 32.

The universal cord 4 is a composite cable allowing insertion of various signal lines which extend from the distal end portion 6 side to the operation section 3 through the inside of the insertion section 2 and which further extend from the operation section 3, and also allowing insertion of a light guide 12 of a light source device (not shown) and an air feeding tube, a water feeding tube and the like extending from an air/water feeding device (not shown), for example.

The endoscope connector 5 includes an electrical connector portion 5a on a side surface portion, and also includes a light source connector portion 5b, an air feeding pipe sleeve 5c and the like. A connector (not shown) of a signal cable connected to a video processor (not shown), which is an external appliance, may be detachably attached to the electrical connector portion 5a. The light source connector portion 5b is connected to the light source device (not shown), which is an external appliance, and the air feeding pipe sleeve 5c is connected to a water feeding port provided on the device.

Next, the configuration of respective portions of the operation section main body 32 will be described in detail.

The operation button group 40 shown in FIG. 1 is configured by including a suction button 41a, and two button switches 42, for example. The suction button 41a is an operation button protruding from a suction valve 41, which is detachably mounted on the operation section main body 32. Arbitrary functions among various functions regarding the endoscope 1 may be assigned to the two button switches 42.

The suction button 41a and the two button switches 42 are arranged in a left-right symmetrical manner on the front side of the operation section main body 32. That is, the suction button 41a is arranged at the center in a left-right width directions of the operation section main body 32 in a manner overlapping the insertion axis O. Also, the two button switches 42 are arranged at left-right symmetrical positions across the insertion axis O, on a distal end side of the suction button 41a.

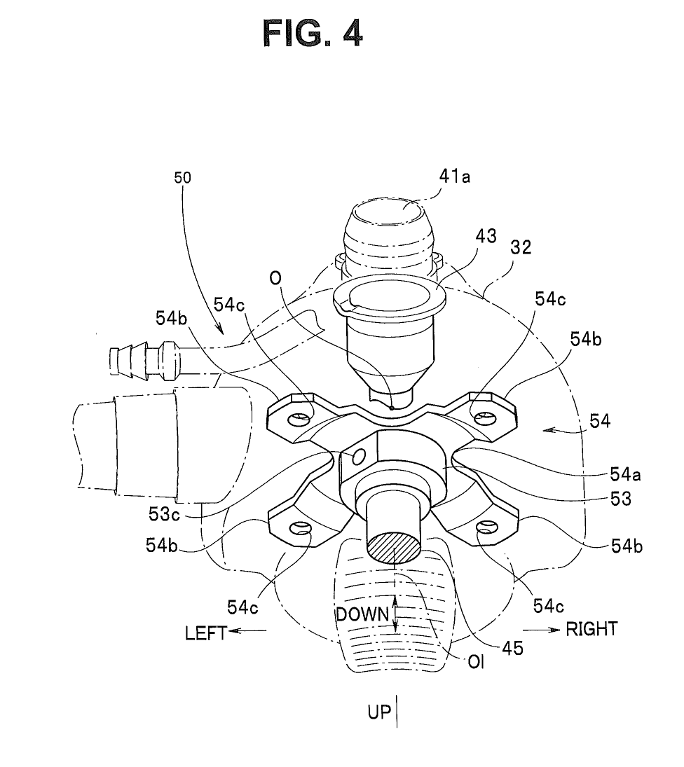

For example, as shown in FIG. 4, a cylinder 43, as a button connection member continuously provided on the suction valve 41, is provided inside the operation section main body 32. The suction valve 41 can be detachably mounted on the cylinder 43. The cylinder 43 is arranged at the center in the left-right width directions of the operation section main body 32 in a manner overlapping the insertion axis O in accordance with the arrangement of the suction button 41a.

For example, the operation lever 45 is configured by a joystick-type lever which can be tilted in any of the directions including up-down/left-right directions. The operation lever 45 is arranged on the back side of the operation section main body 32, at a position where the operation lever 45 becomes left-right symmetrical. In the present embodiment, the operation lever 45 is arranged at the center in the left-right width directions of the operation section main body 32 in a manner overlapping the insertion axis O.

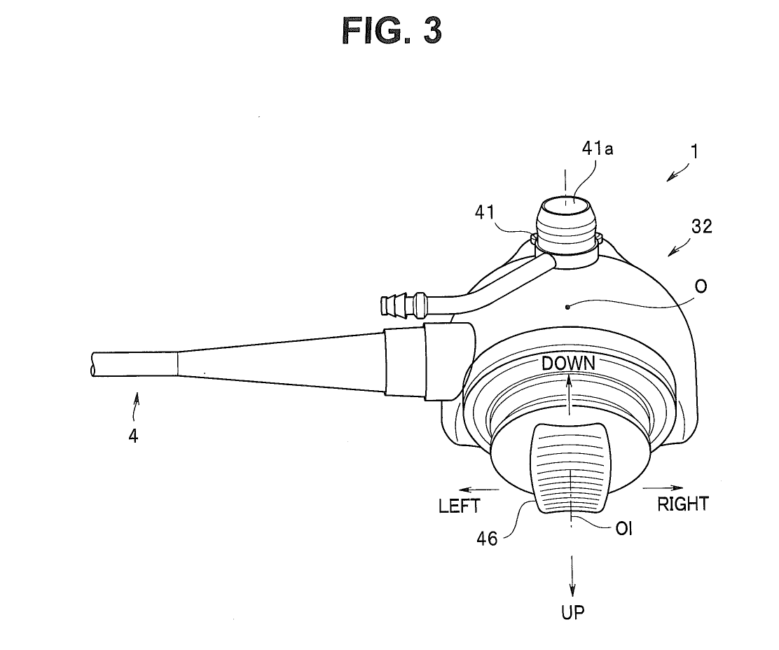

For example, as shown in FIG. 3, with respect to the tilt direction of the operation lever 45, the left-right directions of tilt operation are defined to be the left-right width directions of the operation section 3, which are directions orthogonal to the insertion axis O, and the up-down directions are defined to be a directions which are orthogonal to the left-right width directions.

More specifically, the tilt directions of the operation lever 45 of the present embodiment are defined as follows, for example. The left side as viewed in FIG. 3 is defined as a left tilt direction for bending the bending portion 7 to the left. Also, the right side as viewed in FIG. 3 is defined as a right tilt direction for bending the bending portion 7 to the right. Moreover, the bottom side as viewed in FIG. 3 is defined as an upward tilt direction for bending the bending portion 7 upward. Moreover, the top side as viewed in FIG. 3 is defined as a downward tilt direction for bending the bending portion 7 downward.

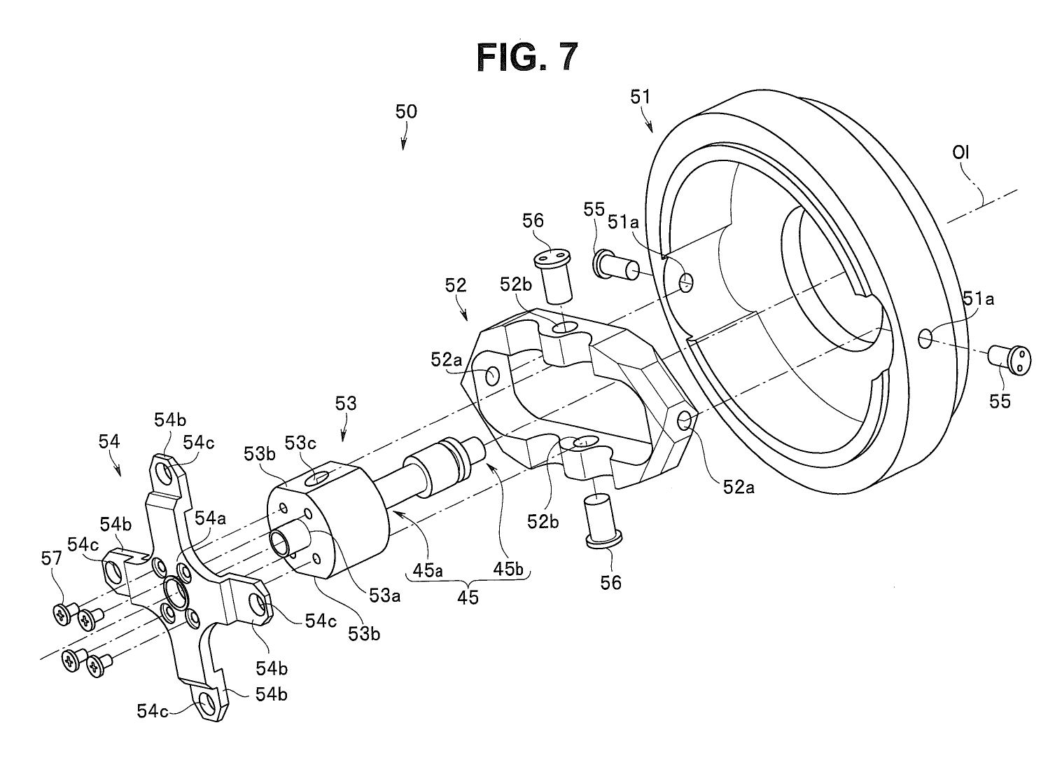

The operation lever 45 is column-shaped (a circular column shape, in the present embodiment), and includes one end portion (see reference sign 45a in FIG. 7) and the other end portion (see reference sign 45b in FIG. 7). A finger rest portion 46 where a thumb or the like of the user is allowed to come into contact is fixedly provided at a tip portion which is the other end portion 45b protruding outward from the operation section 3.

On the other hand, a bending operation mechanism 50 is provided inside the operation section 3, on a one end portion 45a side of the operation lever 45. The operation lever 45 is capable of bending the bending portion 7 in a desired direction by a pulling operation on each operation wire 23 by the bending operation mechanism 50.

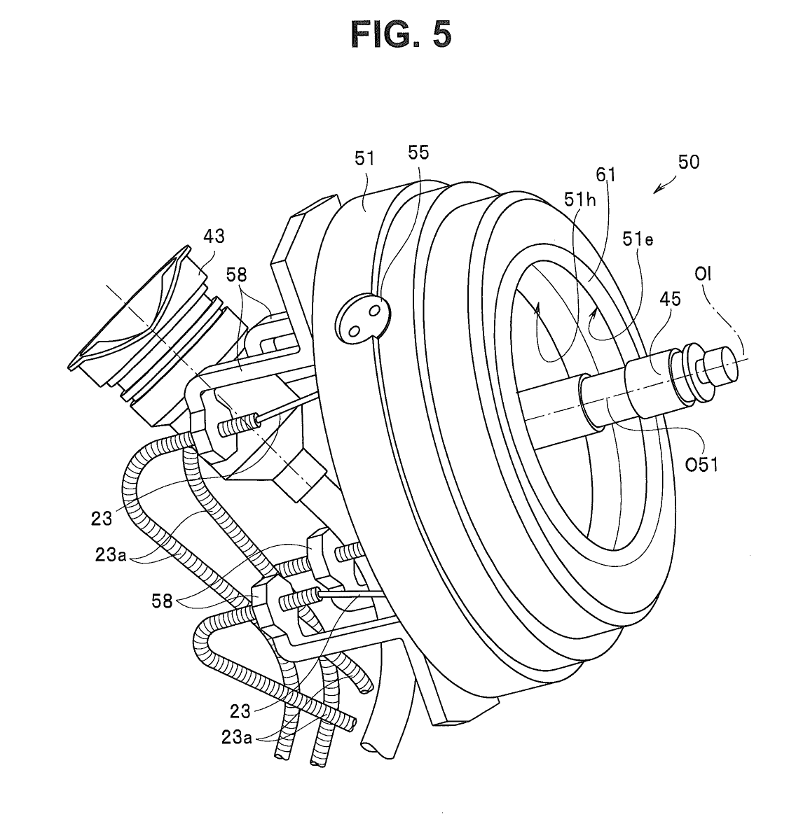

That is, the endoscope operation unit for bending the bending portion 7 of the endoscope 1 is configured from the operation lever 45, the bending operation mechanism 50, and each operation wire 23. Note that the operation lever 45 of the present embodiment is formed by a magnetic body that generates attraction by a magnet described below (see reference sign 61 in FIG. 5).

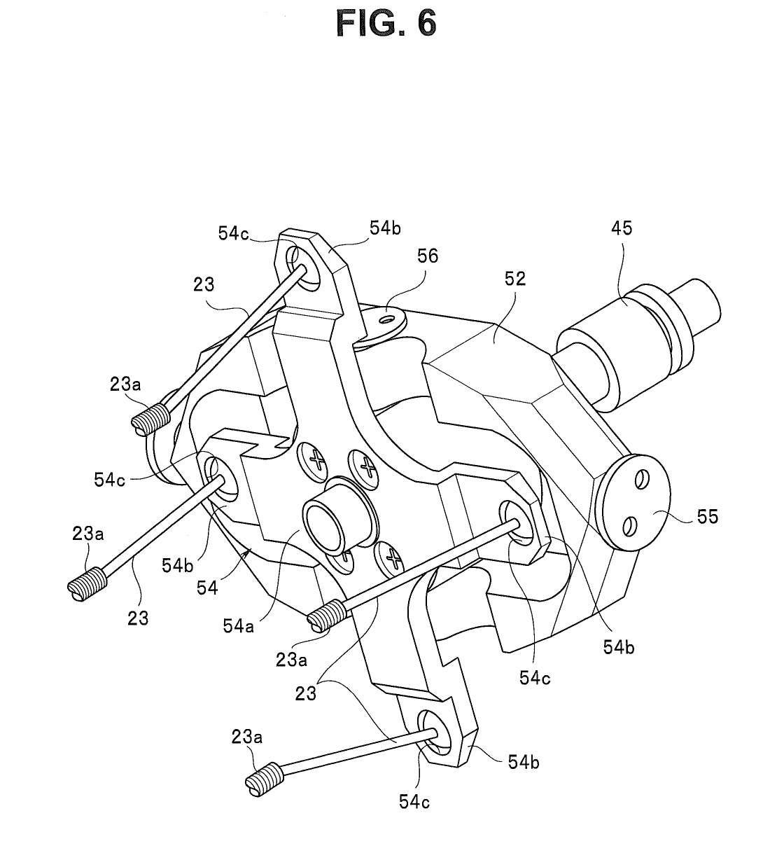

As shown in FIGS. 5, 6 and 7, the bending operation mechanism 50 is configured by including a housing 51, a rotating frame 52, a fixing member 53, and a wire pulling member 54.

The housing 51 is a base section having a substantially cylindrical shape, and includes a through hole 51h where a mid-portion of the operation lever 45 is arranged. The rotating frame 52 is rotatably (in other words, swingably) and pivotally supported inside the through hole 51h of the housing 51. The fixing member 53 is rotatably and pivotally supported inside the rotating frame 52. The wire pulling member 54 is fixedly provided on the fixing member 53.

An annular magnet 61 is disposed on the housing 51. An inner edge of the annular magnet 61 is an inner circumferential portion 51e of the through hole 51h of the housing 51, and the other end portion 45b of the operation lever 45 comes close to the inner edge when the lever 45 is tilted.

Note that shaft holes 51a that face each other are provided piercing a circumferential wall of the housing 51.

The rotating frame 52 is formed from a frame body having a substantially rectangular shape, for example. A pair of screw holes 52a that face each other pierce the rotating frame 52 at the center of both end portions in a long side direction. Also, a pair of shaft holes 52b that face each other pierce the rotating frame 52 at the center of both end portions in a short side direction.

A screw 55 is inserted in each shaft hole 51a of the housing 51. The rotating frame 52 is rotatably and pivotally supported by the housing 51 by the screw 55 being screwed into each screw hole 52a.

The fixing member 53 is formed from a member having a substantially cylinder shape. A fitting hole 53a pierces a center portion of the fixing member 53. The proximal end side of the operation lever 45 is fitted and coupled to the fitting hole 53a. A pair of flat portions 53b that face each other are formed to a circumferential portion of the fixing member 53. Screw holes 53c that face each other (only one of the screw holes 53c is shown in FIG. 7) pierce the flat portions 53b.

A screw 56 is inserted through each shaft hole 52b of the rotating frame 52. The fixing member 53 is rotatably and pivotally supported by the rotating frame 52 by the screw 56 being screwed with each screw hole 53c.

The operation lever 45 coupled to the fixing member 53 is enabled to tilt in an arbitrary direction by the fixing member 53 being supported by the housing 51 through the rotating frame 52 in the above manner.

The wire pulling member 54 is formed from a cross-shaped plate member having arm portions 54b extending in four different directions. An angle formed by adjacent arm portions 54b of the wire pulling member 54 is set to 90 degrees. A center portion 54a of the wire pulling member 54 is fixed to the fixing member 53 by screws 57. That is, the operation lever 45 is coupled to the wire pulling member 54 via the fixing member 53. That is, the four arm portions 54b extend from the center portion 54a sideways and crosswise.

A wire fixing hole 54c pierces a distal end side of each arm portion 54b. The proximal end side of each operation wire 23 extending from the insertion section 2 side is fixed to each wire fixing hole 54c. The wire pulling member 54 is capable of pulling a predetermined operation wire 23 by a predetermined amount of pulling by being caused to swing according to a tilted state of the operation lever 45.

Note that the angle formed by the adjacent arm portions 54b is not limited to 90 degrees, and may be arbitrarily changed in the range of ±30 degrees from 90 degrees on the side of the center portion 54a, for example.

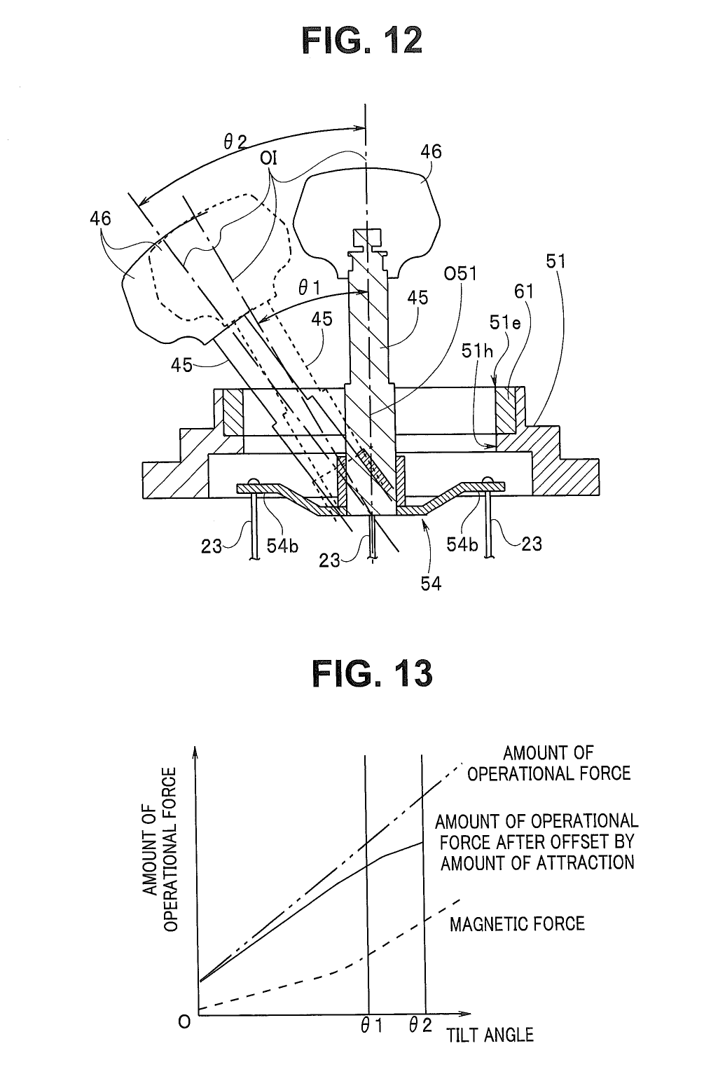

In the present embodiment, the above annular magnet 61 and the operation lever 45 are a bending operation support mechanism configured to reduce the amount of operational force. Note that FIG. 12 shows a case where the operation lever 45 is tilted to the distal end side, for example.

When the bending portion 7 is in a neutral state where the bending portion 7 is straight without being bent, a center axis O1 of the operation lever 45 and a center axis O51 of the through hole 51h coincide with each other.

At the time of bending the bending portion 7 of the endoscope 1, the user tilts the operation lever 45. Then, the operation lever 45 is tilted relative to the center axis O51 of the through hole 51h, and the other end portion 45b of the lever 45 is brought close to the annular magnet 61 provided in the housing 51.

A graph shown by a dashed-two dotted line in FIG. 13 indicates a relationship between a tilted position of the operation lever 45 and the amount of operational force with respect to a configuration where the annular magnet 61 is not provided in the housing 51. The amount of operational force for tilting the operation lever 45 is increased as a tilt angle is increased. In other words, the amount of force for bending the bending portion 7 is increased as a bent angle of the bending portion 7 is increased.

A graph shown by a broken line in FIG. 13 indicates a relationship between a tilted position of the operation lever 45 and the attraction of the magnetic force of the annular magnet 61 provided in the housing 51.

The attraction of the magnetic force of the magnet 61 acting on the operation lever 45 is increased as the tilt angle of the lever 45 is increased. In other words, the attraction generated by the magnetic force of the magnet 61 on the operation lever 45 is increased as the lever 45 gets closer to the magnet 61.

Accordingly, the amount of operational force for the operation lever 45 according to the configuration where the annular magnet 61 is provided in the housing 51 takes a value, as indicated by a solid line in FIG. 13, obtained by offsetting the amount of operational force indicated by the dashed-two dotted line by the attraction generated on the operation lever 45 by the magnetic force of the magnet 61, indicated by the broken line.

As described above, the annular magnet 61 is provided at a predetermined position of the housing 51, and the operation lever 45 is formed by a member on which attraction is generated by the magnetic force of the magnet 61. Accordingly, a user may perform a tilt operation of the operation lever 45 while obtaining attraction that is generated by the magnetic force of the magnet 61, and reduction in the amount of operational force may thereby be realized.

Note that the magnetic force indicated by the broken line is changed by the magnitude of the amount of magnetism of the magnet 61. That is, desirable tilt operability can be realized by appropriately setting the amount of magnetism of the magnet.

That is, as shown in FIGS. 11 and 12, according to the endoscope 1 of the present embodiment, the amount of operational force is reduced by the attraction generated by the magnetic force of the magnet 61 in a state where the operation lever 45 is tilted by an angle θ1. Then, the attraction is further increased when the operation lever 45 is tilted toward an angle θ2, and the amount of operational force is increased more gradually as the attraction is increased.

Accordingly, at the time of tilting the operation lever 45, the user can perform the tilt operation on the operation lever 45 in a state where the amount of operational force for the lever 45 is reduced due to the attraction generated on the operation lever 45.

As described above, according to the endoscope 1 provided with the magnet 61 and the operation lever 45, the operation force for bending the bending portion 7 by the operation lever 45 is reduced and fatigue of the user is prevented, and also, a fine bending operation can be realized due to increased bending operability.

Note that in a state where the operation lever 45 is tilted by the angle θ2, a gap is formed between a side circumferential surface of the lever 45 and the inner circumferential portion 51e. A bending boot (not shown) configured to maintain water-tightness between the operation lever 45 and the operation section main body 32 is arranged in the gap.

The bending operation mechanism 50 provided with the bending operation support mechanism configured in the above manner is arranged inside the operation section main body 32, while facing the cylinder 43 with one behind the other.

Each arm portion 54b of the bending operation mechanism 50 is arranged at a position rotated around the center axis O1 of the operation lever 45 in a range of 30 degrees to 60 degrees (for example, at a position rotated by 45 degrees) relative to the upward-downward/left-right tilt directions defined for the operation lever 45. Accordingly, as shown in FIG. 4, for example, the bending operation mechanism 50 is disposed with the cylinder 43 facing between two adjacent arm portions 54b of the wire pulling member 54.

Furthermore, as shown in FIG. 5, for example, two stays 58 extending on each of both sides of the cylinder 43 are provided on the housing 51 of the bending operation mechanism 50. One end portions of guide coils 23a are fixed to the stays 58. The guide coils 23a are routed around the cylinder 43 to prevent interference. Respective operation wires 23 are inserted through the guide coils 23a. As a result, each operation wire 23 is routed without interfering with the cylinder 43.

With respect to such a configuration, a user grasps the grasping portion 31 of the operation section 3, and tilts the operation lever 45 in the left tilt direction by the thumb of the grasping hand. Then, the operation wires 23 coupled to the two arm portions 54b positioned in the right tilt direction are mainly pulled. Then, the two operation wires 23 positioned on a left side of a bending direction are mainly pulled inside the bending portion 7, and the bending portion 7 is bent to the left.

Because the bending operation support mechanism is provided, the user can perform a fine bending operation at the time of tilting the operation lever 45 in the left tilt direction, and also, fatigue of performing the bending operation is prevented.

Furthermore, the user grasps the grasping portion 31 of the operation section 3, and tilts the operation lever 45 in the right tilt direction by the thumb of the grasping hand. Then, the operation wires 23 coupled to the two arm portions 54b positioned in the left tilt direction are mainly pulled. Then, the two operation wires 23 positioned on a right side of the bending direction are mainly pulled inside the bending portion 7, and the bending portion 7 is bent to the right.

Because the bending operation support mechanism is provided, the user can perform a fine bending operation at the time of tilting the operation lever 45 in the right tilt direction, and also, fatigue of performing the bending operation is prevented.

Furthermore, the user grasps the grasping portion 31 of the operation section 3, and tilts the operation lever 45 in the upward tilt direction by the thumb of the grasping hand. Then, the operation wires 23 coupled to the two arm portions 54b positioned in the downward tilt direction are mainly pulled. Then, the two operation wires 23 positioned on a top side of the bending direction are mainly pulled inside the bending portion 7, and the bending portion 7 is bent upward.

Because the bending operation support mechanism is provided, the user can perform a fine bending operation at the time of tilting the operation lever 45 in the upward tilt direction, and also, fatigue of performing the bending operation is prevented.

Moreover, the user grasps the grasping portion 31 of the operation section 3, and tilts the operation lever 45 in the downward tilt direction by the thumb of the grasping hand. Then, the operation wires 23 coupled to the two arm portions 54b positioned in the upward tilt direction are mainly pulled. Then, the two operation wires 23 positioned on a bottom side of the bending direction are mainly pulled inside the bending portion 7, and the bending portion 7 is bent downward.

Because the bending operation support mechanism is provided, the user can perform a fine bending operation at the time of tilting the operation lever 45 in the downward tilt direction, and also, fatigue of performing the bending operation is prevented.

Moreover, when the user guides the index finger or the like of the hand grasping the operation section 3 to the operation button group 40 along the guide recess portion 32a and presses the suction button 41a, for example, a suction operation by the endoscope 1 is performed.

The grasping portion 31 of the operation section 3 has a left-right symmetrical shape, and the suction button 41a and the operation lever 45 are disposed facing each other at the center in the left-right width direction of the operation section main body 32 of the operation section 3. The user is thereby allowed to grasp the operation section 3 in the same manner by either of the left and right hands, and also, to operate the suction button 41a and the operation lever 45 with the same operability.

Each arm portion 54b is disposed at a position rotated around the center axis O1 of the operation lever 45 by a predetermined angle from upward-downward/left-right tilt directions defined for the operation lever 45. Accordingly, the arm portions 54b and the like may be prevented from interfering with the cylinder 43.

Particularly, the arm portions 54b, the operation wires 23 and the like moving in coordination with the operation lever 45 may be prevented from interfering with the cylinder 43 when the operation lever 45 is tilted in the upward-downward tilt directions. Accordingly, the suction button 41a and the operation lever 45 may be disposed facing each other with one behind the other at the center in the left-right width directions of the operation section 3 without greatly separating the wire pulling member 54 (bending operation mechanism 50) away from the cylinder 43, and also, the same operability may be realized for both the left and the right hands without increasing the size of the operation section 3.

Moreover, also in a case where the tilt directions of the operation lever 45 and an extending directions of the arm portions 54b are made different around the center axis O1 of the operation lever 45 on an operation section 3 side, the operation wires 23 routed in the bending portion 7 are substantially correspondingly routed at positions rotated around the insertion axis O by set angles relative to the upward-downward/left-right bending directions of the bending portion 7. Accordingly, the upward-downward/left-right bending directions of the bending portion 7 may be easily made to coincide with the upward-downward/left-right tilt directions defined for the operation lever 45.

Moreover, according to such an arrangement, the operation wires 23 do not have to be routed in the left-right directions inside the bending portion 7. Accordingly, for example, bending operations in the up-down/left-right directions may be realized by using a layout inside the bending portion where the image pickup section 11 and the treatment instrument insertion channel 13, which are large members, are horizontally arranged and which is capable of bending only in the up-down directions, without increasing the outer diameter.

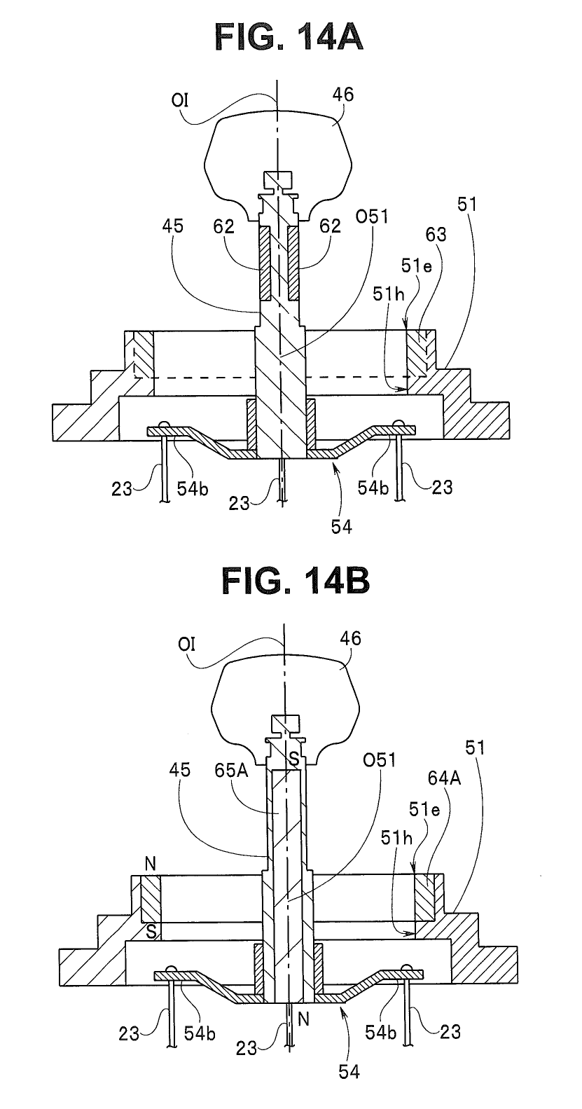

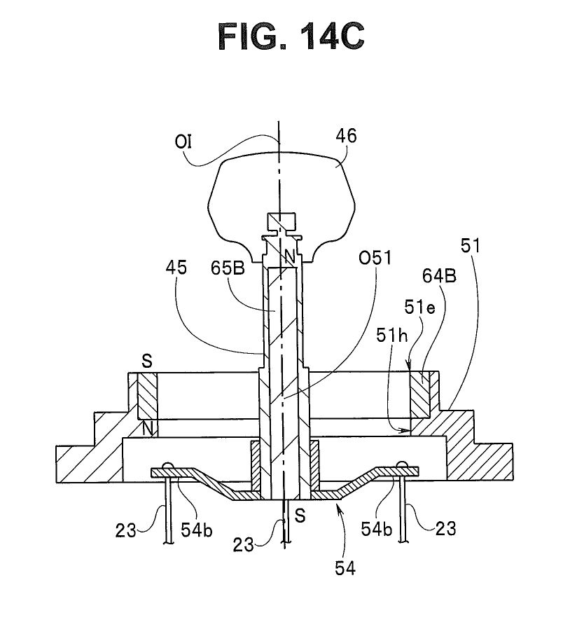

Note that in the embodiment described above, the annular magnet 61 is disposed on the housing 51, and the operation lever 45 is formed by a magnetic body on which attraction is generated by the magnetic force of the magnet 61. However, as shown in FIG. 14A, a U-shaped magnet 62, which is branched into two for example, may be disposed on the operation lever 45, and the housing 51 may be formed by a magnetic body on which attraction is generated by the magnetic force of the magnet 62, or a magnetic body 63, which is indicated by a broken line and which is annular, like the magnet 61, and on which attraction is generated by the magnetic force of the magnet 62 may be disposed on the housing 51.

The same actions and effects as the actions and effects of the embodiment described above may be achieved by the configuration. Note that other structures and actions are the same as the structures and actions of the embodiment described above, and description is omitted by denoting the same members with the same reference signs. Furthermore, instead of disposing the U-shaped magnet 62, which is branched into two, on the operation lever 45, an annular magnet which is divided into two may be disposed on the operation lever 45, and the housing 51 may be formed by a magnetic body on which attraction is generated by the magnetic force of the annular magnet.

Moreover, as shown in FIG. 14B or 14C, an annular magnet 64A, 64B may be disposed on the housing 51, and a bar magnet 65A, 65B may be disposed on the operation lever 45. Note that as shown in FIG. 14B, an N pole of the annular magnet 64A is provided on an operation section opening side, and an S pole is provided inside the operation section.

In contrast, an S pole of the bar magnet 65A is provided on the other end portion side, and an N pole is provided on the one end portion side. As shown in FIG. 14C, an N pole of the annular magnet 64B is provided inside the operation section, and an S pole is provided on the operation section opening side. In contrast, an S pole of the bar magnet 65B is provided on the one end portion side, and an N pole is provided on the other end portion side.

According to the configuration, the same actions and effects as the actions and effects of the embodiment described above may be achieved. Note that other structures and actions are the same as the structures and actions of the embodiment described above, and description is omitted by denoting the same members with the same reference signs.

Note that the present invention is not limited to the embodiment described above, and various changes and alterations may be made within the scope of the present invention. For example, the embodiment described above describes an example of applying the present invention to an endoscope for bronchus. However, the present invention is not limited to such a case, and may also be applied to an endoscope for urinary organs, for example.

Furthermore, it is needless to say that the tilt directions defined for the operation lever are not limited to the directions described above, and that the operation buttons are not limited to the suction button and the like.

What is claimed is:

1. An operation unit of an endoscope, the operation unit comprising:
an insertion section that is insertable into a subject, and that includes a bending portion that can be bent in a predetermined direction from a longitudinal axis;
an operation section that is provided continuously to a proximal end of the insertion section;
a plurality of operation wires that are inserted inside the insertion section and the operation section, and that are configured to bend the bending portion by being pulled or slackened;
an operation lever that is provided on the operation section, relative to one end of the lever, other end of the operation lever being freely tiltable in the predetermined direction of the bending portion;
a wire pulling member that is swingably provided inside the operation section, the one end of the operation lever being coupled to a center portion of the wire pulling member, proximal end sides of the wires being coupled to respective distal end sides of a plurality of arm portions extending sideways from the center portion;
a cylindrical base section that is fixedly provided at a predetermined position of the operation section, and that includes a through hole where a mid-portion of the operation lever is arranged and an inner circumferential portion to which the other end of the operation lever comes close when the operation lever is tilted in the predetermined direction;
a magnet that is provided on one of the base section and the other end of the operation lever; and
a member on which attraction is generated by a magnetic force of the magnet, the member being provided on the other end of the operation lever when the magnet is provided on the base section and provided on the base section when the magnet is provided on the other end of the operation lever.

2. The operation unit of an endoscope according to claim 1, wherein
the magnet having an annular shape is disposed on the base section, and
a magnetic body is disposed on a side of the other end of the operation lever.

3. The operation unit of an endoscope according to claim 1, wherein
the magnet is disposed on a side of the other end of the operation lever, and
a magnetic body is disposed on the base section.

4. The operation unit of an endoscope according to claim 1, wherein
the magnet is disposed on a side of the other end of the operation lever, and
a second magnet, poles of which are opposite to poles of the magnet, is disposed on the base section.

5. The operation unit of an endoscope according to claim 1, wherein
the endoscope includes an image pickup section configured to pick up an image of the subject, and
an image pickup direction of the image pickup section can be freely changed by a tilt operation of the operation lever.
